(19) United States
(12) Patent Application Publication
(10) Pub. No.: US 2014/0295574 A1
(43) Pub. Date: Oct. 2, 2014

(54) Method of Fabricating Testing Reagent Carrier through Ionizing Radiation

(71) Applicant: Institute of Nuclear Energy Research, Atomic Energy Council, Executive Yuan, R.O.C., Taoyuan County (TW)

(72) Inventors: Kuan-Yin Chen, Taoyuan County (TW); Chun-Ying Chen, Taoyuan County (TW); Meng-Jun Fu, Taoyuan County (TW); Feng-Huei Lin, Taipei City (TW); Chia-Ching Liu, Taipei City (TW)

(73) Assignee: Institute of Nuclear Energy Research, Atomic Energy Council, Executive Yuan, R.O.C., Taoyuan County (TW)

(21) Appl. No.: 13/958,845

(22) Filed: Aug. 5, 2013

(30) Foreign Application Priority Data
Apr. 2, 2013 (TW) 102111981

(51) Int. Cl. G01N 33/574

(52) U.S. Cl. CPC G01N 33/574
USPC 436/501

(57) ABSTRACT

A method is provided for modifying a radioactive carbon nanotube (CNT) carrier. Magnetic molecules are used. The modified CNT is highly specified to disease. Surface of the CNT has functional grafts for catching antigen/antibody. Antigen/antibody thus caught on the surface is increased in number. Thus, the present invention improves sensitivity and accuracy of disease detection and greatly saves cost. The present invention can be applied for sample purification or massive disease detection.

### TECHNICAL FIELD OF THE INVENTION

The present invention relates to a testing reagent carrier; more particularly, relates to using PEG to modify a surface of a carbon nanotube (CNT) through chemical bonding for obtaining a testing reagent for labeling specific cancer with magnetic molecules on the surface of the CNT.

### DESCRIPTION OF THE RELATED ARTS

It is not easy for a general material to adhere a specific antigen/antibody of a disease. Especially, the adhesion is not strong and the antigen/antibody may be easily lost or the protein may be thus harmed to lose its activity. A few methods are revealed for strengthening the adhesion, such as developing a carrier used as a way for separation and purification. Therein, a technology of a magnetic carrier obtained after functionalizing a CNT is revealed. Functional groups are formed on surface of the CNT to affect the adhesion. After an antigen or antibody is combined with the functionalized CNT, extra magnetic molecules are added to be adhered on the nanoparticles by heating for easily moving them to another place or into another status under effect of a magnetic field.

Nano carbon was found on fabricating Bucky ball (C-60) in 1991. CNT is excellent in physical, chemical and material characteristics, like light weight, high ductility, high flexibility, big surface area, high thermal transmission, etc. Hence, CNT is highly welcomed for various applications. Furthermore, the magnetic carrier is applied for separating a target by using magnetic field, which is widely used in medical diagnosis, DNA and RNA purification, protein and enzyme fixation, immue analysis, environmental analysis, magnetic fluid, etc. For example, after being combined with a specific antigen/antibody, the magnetic carrier obtains high bio-selectivity and can be adhered to specific DNA, RNA or antigen/antibody of a disease or virus.

Accordingly, in noncytopathic (NCP) period before causing cancer, disease detection and diagnosis help early treatment for improving cure of cancer patient. Clinically, disease detection and diagnosis mainly rely on immue analysis of inter-reactions of antibody-antigen, including enzyme-linked immunosorbent assay (ELISA), chemiluminesce, polymerase chain reaction (PCR), surface plasma resonance (SPR), electrochemical QCM (EQCM), immuno-PCR, etc. For example, a commercial magnetic carrier uses bio-glass matrix; yet, it will be hydrolyzed in water, not to mention its stability will be lost on changing its pH value. Furthermore, the OH group on the magnetic carrier can be easily combined with water molecule to cause a problem of non-specific binding, which reduces its sensitivity.

Hence, the prior arts do not fulfill all users' requests on actual use.

### SUMMARY OF THE INVENTION

The main purpose of the present invention is to use PEG to modify a surface of a CNT through chemical bonding for obtaining a testing reagent for labeling specific cancer with magnetic molecules on the surface of the CNT.

To achieve the above purpose, the present invention is a method of fabricating a testing reagent carrier through ionizing radiation, comprising steps of: (a) processing a CNT with an acid and illuminating the CNT through ionizing radiation of cobalt(Co)-60 to modify a surface of the CNT for obtaining functional grafts on the surface; (b) adding a plurality of magnetic molecules to be illuminated through ionizing radiation of Co-60 for adhering the magnetic molecules on the surface; and (c) using PEG to modify the surface through chemical bonding for forming functional grafts on the surface to easily catch an antibody or antigen. Accordingly, a novel method of fabricating a testing reagent carrier through ionizing radiation is obtained.

### BRIEF DESCRIPTIONS OF THE DRAWINGS

The present invention will be better understood from the following detailed description of the preferred embodiment according to the present invention, taken in conjunction with the accompanying drawings, in which

FIG. 1 is the flow view showing the preferred embodiment according to the present invention;

FIG. 2 is the structural view showing the magnetic nano composite; and

FIG. 3 is the view showing the use of the magnetic nano composite.

### DESCRIPTION OF THE PREFERRED EMBODIMENT

The following description of the preferred embodiment is provided to understand the features and the structures of the present invention.

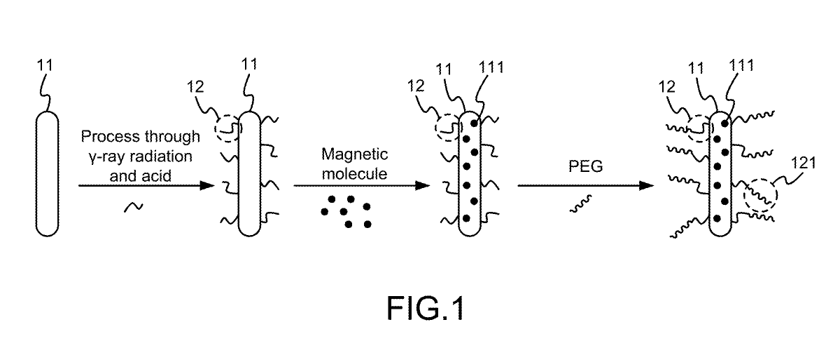

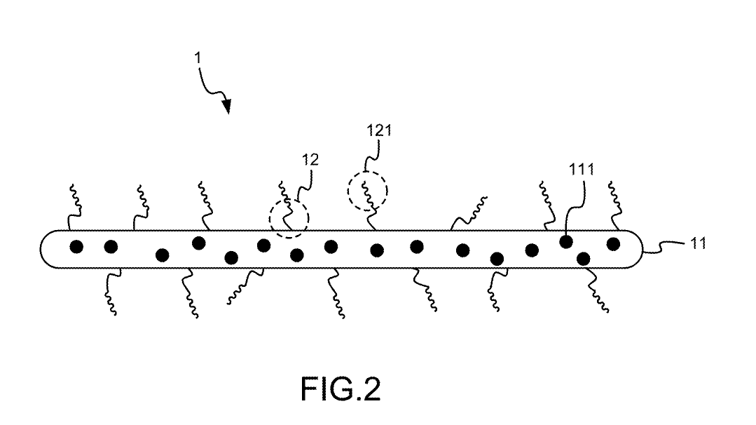

Please refer to FIG. 1 and FIG. 2, which are a flow view showing a preferred embodiment according to the present invention; and a structural view showing a magnetic nano composite. As shown in the figures, the present invention is a method of fabricating a testing reagent carrier through ionizing radiation, comprising the following steps:

(a) A carbon nanotube (CNT) 11 is processed with an acid and is illuminated through ionizing radiation of cobalt(Co)-60 to modify a surface of the CNT 11 for forming functional grafts on the surface.

(b) A plurality of magnetic molecules 111 is added to be illuminated through ionizing radiation of Co-60 so that the magnetic molecules 111 are adhered on the surface.

(c) Polyethylene glycol (PEG) 121 is used to modify the surface through chemical bonding to obtain a modified CNT of a magnetic nano composite 1, so that an antibody or antigen can be easily captured by the magnetic nano composite 1. Therein, the modified CNT comprises a magnetic CNT and a plurality of functional grafts 12; the magnetic CNT is the CNT 11 distributed with the magnetic molecules 111; the magnetic molecule 111 is Fe²⁺, which can be synthesized into Fe₃O₄; the functional grafts 12 are distributed on the surface of the CNT 11 and adhered with PEG 121 for combining active molecules 2 on the surface of the CNT 11; the functional graft 12 has a functional group and the functional group is —COOH, —NH2, —SH, —OH, —COH or —COO—.

On using the present invention, a CNT 11 is processed with an acid of nitric acid, sulphuric acid or acetic acid; and is illuminated through ionizing radiation of Co-60. Functional grafts 12 are thus formed on the CNT 11, which can be functional groups of COOH. Then, a plurality of magnetic molecule 111 is added to the CNT 11 and is illuminated through ionizing radiation of Co-60 to be adhered on a surface of the CNT 11. At last, PEG 121 is used to modify the surface for improving water solubility, bio-compatibility and for that specificity on target is achieved by avoiding being caught by protein in serum. Each CNT 11 is magnetic and, at the same time, specific binding is enhanced by PEG 121. Thus, the magnetic nano composite 1 fabricated through ionizing radiation can be used as a testing nano reagent for specifically labeling cancers, where testing sensitivity is improved and error signals are reduced for diagnosing specific diseases.

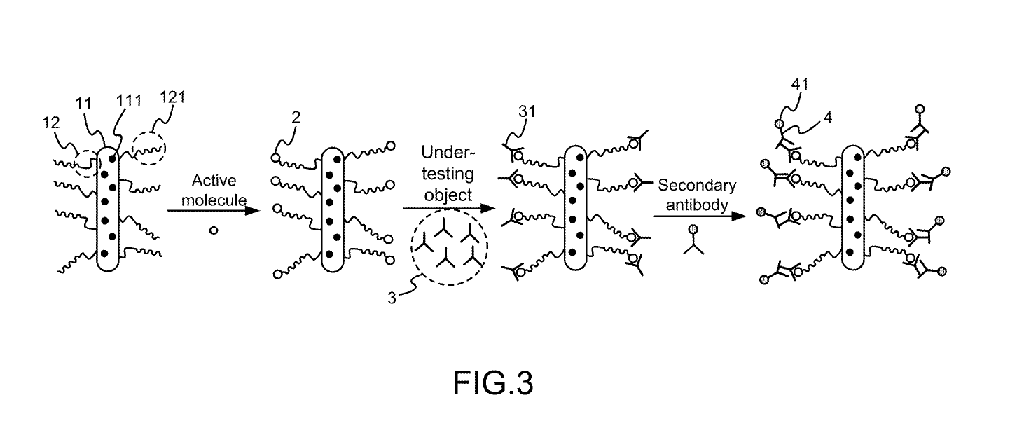

Please refer to FIG. 3, which is a view showing use of a magnetic nano composite. As shown in the figure, a magnetic nano composite 1 fabricated according to the present invention is applied for clinical mass regular in-vitro cancer quantitative measurement, comprising the following steps:

(a) The magnetic nano composite 1 is provided, which is grafted with PEG and is combined with a plurality of active molecules 2 on outside surface for catching a plurality of under-testing objects 31. The active molecule 2 is an antigen, a nucleic acid, an oligonucleotide, a protein, a saccharide or an antibody.

(b) A sample 3 is added to the magnetic nano composite 1. The under-testing objects 31 in the sample 3 are specifically reacted with the active molecules 2 to be caught on the outside surface of the magnetic nano composite 1.

(c) A secondary antibody 4 labeled with a radioactive material 41 is added for reaction, so that the secondary antibody 4 labeled with the radioactive material 41 labels the under-testing objects 31 and the labeled under-testing objects 31 are used as indexes for signal detection.

On using the magnetic nano composite 1 as a carrier, an antigen (the active molecule) 2 is combined on a surface of the CNT 11 by the functional grafts 12 and PEG 121 on the functional grafts 12. Then, by a magnet put under a container, the magnetic molecules 111 in the magnetic nano composite 1 are reacted with a magnetic field to be moved toward and gathered at the magnet. After removing un-reacted antigen 2 by washing, the magnetic nano composite 1 containing the antigen 2 purely is obtained. Then, a sample 3 (i.e. serum of a cancer patient) is added in. By using specificity of the magnetic nano composite 1 on adhering to a specific disease, the under-testing objects 31 of the antigen, which is a primary antibody, are grafted on the magnetic nano composite 1. Therein, in the same way, a magnet field is used to attract and gather the magnetic nano composite 1 and the other un-attracted material in the sample 3 is separated and removed. At last, the magnetic nano composite 1 grafted with the primary antibody is grafted with the secondary antibody 4 to be attracted and gathered by a magnetic field. The un-attracted secondary antibody 4 is separated and removed. Then, signal of the radioactive material 41 (e.g. iodine(I)-125) on the secondary antibody 4 is detected.

Therein, if the serum of the cancer patient has no corresponding primary antibody, the antigen 2 on the magnetic nano composite 1 would not reacted and the same to the secondary antibody 4, so that false positive is avoided on detecting the signal of the radioactive material.

To sum up, the present invention is a method of fabricating a testing reagent carrier through ionizing radiation, where a CNT is modified for obtaining functional grafts on its surface; a plurality of magnetic molecules is added to be adhered on the surface; and PEG is used to obtain a modified CNT of a magnetic nano composite for easily catching an antibody or antigen.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method of fabricating a testing reagent carrier through ionizing radiation, comprising steps of:
(a) processing a carbon nanotube (CNT) with an acid and illuminating said CNT through ionizing radiation of cobalt(Co)-60 to modify a surface of said CNT to obtain functional grafts on said surface of said CNT;
(b) adding a plurality of magnetic molecules to be illuminated through ionizing radiation of Co-60 to adhere said magnetic molecules on said surface of said CNT; and
(c) obtaining polyethylene glycol (PEG) to modify said surface of said CNT through chemical bonding to obtain a modified CNT,
wherein said modified CNT comprises
a magnetic CNT; and
a plurality of functional grafts, said functional grafts being distributed on said surface of said CNT and adhered with PEG.

2. The method according to claim 1,
wherein, in step (b), said magnetic molecule is Fe²⁺.

3. The method according to claim 2,
wherein, said magnetic molecule of Fe²⁺ is synthesized into Fe₃O₄.

4. The method according to claim 1,
wherein said functional graft has a functional group and said functional group is selected from a group consisting of —COOH, —NH₂, —SH, —OH, —COH and —COO—.

5. The method according to claim 1,
wherein said acid is selected from a group consisting of nitric acid, acetic acid and sulphuric acid.

6. The method according to claim 1,
wherein said active molecule is selected from a group consisting of an antigen, a nucleic acid, an oligonucleotide, a protein, a saccharide and an antibody.

7. The method according to claim 1,
wherein said modified CNT comprises a method to clinically diagnose cancer through a quantitative measurement and said method comprises steps of:
(a) obtaining said modified CNT to be combined with a plurality of active molecules,
wherein said modified CNT is a magnetic nano composite grafted with PEG and combined with said active molecules on an outer surface of said magnetic nano composite;
(b) adding a sample to said magnetic nano composite to catch under-testing objects in said sample by a specific response between said under-testing objects and said active molecules on said magnetic nano composite; and
(c) adding a secondary antibody labeled with a radioactive material to react with said under-testing objects to obtain said labeled under-testing objects as an index.

8. The method according to claim 7,
wherein said sample is a serum of a cancer patient.

9. The method according to claim 7,
wherein said under-testing object is a primary antibody.

10. The method according to claim 7,
wherein said radioactive material is iodine(I)-125.
